(19) United States
(12) Patent Application Publication
(10) Pub. No.: US 2017/0143871 A1
(43) Pub. Date: May 25, 2017

(54) Gold Nanorod Incorporated Gelatin based Hybrid Hydrogels for Cardiac Tissue Engineering and Related Methods

(71) Applicant: ARIZONA BOARD OF REGENTS ON BEHALF OF ARIZONA STATE UNIVERSITY, SCOTTSDALE, AZ (US)

(72) Inventors: Mehdi Nikkhah, Scottsdale, AZ (US); Ali Navaei, Tempe, AZ (US)

(73) Assignee: ARIZONA BOARD OF REGENTS ON BEHALF OF ARIZONA STATE UNIVERSITY, SCOTTSDALE, AZ (US)

(21) Appl. No.: 15/336,511

(22) Filed: Oct. 27, 2016

Related U.S. Application Data
(60) Provisional application No. 62/248,202, filed on Oct. 29, 2015.

(51) Int. Cl. A61L 27/22; A61L 27/04; A61L 27/52

(52) U.S. Cl. CPC A61L 27/222; A61L 27/52; A61L 27/047; A61L 2430/20; A61L 2400/12

(57) ABSTRACT

A cell construct including GelMA-GNR hybrid hydrogels formed of GelMA and gold nanorods; and where the gold nanorods are embedded in the GemMA hydrogel.

### RELATED APPLICATIONS

This application claims the benefit of priority of U.S. Provisional Application No. 62/248,202, filed Oct. 29, 2015, the contents of which are incorporated herein by reference in their intirety.

### BACKGROUND

Myocardial infarction (MI) is one the highest causes of mortality among cardiovascular diseases (CVD) in the United States causing approximately one death per minute. Due to limited availability of donors and high complications associated with heart transplantation, cell-based therapy and cardiac tissue engineering have been considered as promising approaches for treatment of MI. Particularly, cardiac tissue engineering have been provided suitable platforms, such as micro and nanoengineered patches, meshes, and cell sheets, capable of mimicking the extra cellular matrix (ECM) of myocardium structure, eventually may recover the tissue or organ loss. In this regard, hydrogel-based scaffolds provide excellent 3D cross-linked matrices for specific applications in cardiac repair and regeneration. The swollen network of hydrogels facilitates nutrient and gas diffusions while resembling the structural complexities of proteins in the myocardial ECM. To date, several synthetic and natural hydrogels, such as collagen, gelatin alginate, Matrigel, and poly(N-isopropylacrylamide) (PNIPAAM), have been synthesized to develop tissue constructs with the purpose of eventually replacing dysfunctional heart muscle. Although, the macporous structure of the hydrogels offers an ECM-like environment to support cell functions, the nanostructural, and electrical properties of the hydrogels are inferior to the characteristics of native myocardium. In other words, inadequate cell adhesion points, and electrically insulated structure of conventional hydrogels ultimately lead to poor patch-myocardium integration.

Recently, several studies have demonstrated that employing electrically conductive nanomaterials enables addressing the shortcomings of conventional hydrogel-based scaffolds. In this regard carbon nanotubes (CNT) have been among well respected conductive nanomaterials for cardiac tissue engineering. CNTs-embedded scaffolds have particularly demonstrated enhanced electrical properties that facilitated electrical signal propagation and cell-cell coupling. While incorporation of CNTs results in superior properties, several controversial cytotoxicity issues have raised numerous concerns for their use in clinical applications. Several techniques such as surface coating and/or functionalization, have been proposed in order to reduce the cytotoxicity level of CNTs. However, these alterations may compromise the electrical properties of the scaffold, which in turn, increases the risk of cytotoxicity. In addition, the low solubility of carbon nanotubes may require complex fabrication procedures in engineering tissue constructs. Therefore, utilizing other nanomaterials with similar electrical characteristics and higher biocompatibility (minimized cytotoxicity) may provide ideal solution for applications in cardiac tissue engineering.

### SUMMARY

A cell construct includes GelMA-GNR hybrid hydrogels formed of GelMA and gold nanorods, and where the gold nanorods are embedded in the GemMA hydrogel.

A method of making a cardiac tissue patch assembly for myocardial regeneration and repair is described herein. The method includes synthesizing gold nanorods, adding gold nanorods to GelMA prepolymer solution and forming GelMA-GNR, and applying UV irradiation to the GelMA-GNR to crosslink the GelMA-GNR.

### BRIEF DESCRIPTION OF THE DRAWINGS

FIGS. 1A-1I illustrate fabrication and characterization of GNRs and GelMA-GNR hybrid hydrogels. 1A) TEM micrographs of synthesized GNRs; 1B) UV-Vis absorbance spectra of GNPs and GNRs, showing longitudinal and transvers surface plasmon resonance (SPR) peaks; 1C) UV-Vis absorbance spectra of pure GelMA and GelMA-GNR hybrid prepolymer solution with 0.5, 1, and 1.5 mg/mL GNRs concentrations; 1D) schematic illustration of GelMA-GNRs hybrid hydrogel construct fabrication procedure (numbers indicate the step sequence); 1E) TEM micrograph showing GNRs locating within a very thin layer of GelMA-GNRs (1.5 mg/mL), (arrow heads pointing GNRs); 1F) optical pictures of hybrids and pure hydrogel constructs, and SEM images illustrating cross-section of GelMA-GNRs hybrids and pure GelMA hydrogel; 1G) impedance of whole hydrogel construct (150 μm thick) decreased as a function of GNRs; 1H) swelling ratio of GelMA-GNR with 1 and 1.5 mg/mL nanorod concentrations decreased considerably compared to pure GelMA (*p<0.05); 1I) porosity percentage of GelMA-GNR hybrid and pure GelMA hydrogels calculated based on SEM image (n=5) demonstrating the GNRs did not changed macroporous architecture of pure GelMA hydrogel.

FIGS. 2A-2E illustrate cardiomyocytes retention, survival, and metabolic activity. 2A) Phase-contrast images and 2B) fraction area of hydrogels covered by cardiomyocytes one day after cell seeding of all hybrid and pure hydrogels demonstrating more uniform and packed cell adhesion profile and significant higher cell retention in GelMA-GNR as compared to pure GelMA hydrogel (*p<0.05); 2C) representative fluorescent images of live (green) and dead (red) cells and 2D) quantified cellular viability at day 1 and 7 of culture showed maintaining high level of cell survival over the whole duration of culture ($, V, §, £p<0.05). 2E) Normalized Alamar Blue fluorescent signal depicting increased cellular proliferation at day 3, day 5, and day 7 of culture. All scale bars are 250 μm.

FIGS. 3A-3C illustrate cytoskeleton organization and alignment of cardiomyocytes. 3A) Z-stack (top-view) fluorescent images of F-actin stained (green) at day 5 of culture for pure GelMA and all hybrid GelMA-GNR hydrogels illustrating highly organized, packed, and uniform distribution, as well as local alignment (with arrows) of actin fibers. DAPI (blue) represents stained nuclei. FFT images (top right insets) demonstrating local alignment of F-actin fibers in hybrid GelMA-GNR construct, in contrast with randomly distributed filament in pure GelMA hydrogel (top-row scale bars represent 50 μm and bottom-row scale bars represent 20 μm). 3B) Cardiomyocytes' nuclei alignment distribution from 0 to 90 degrees span representing averagely similar number of aligned nuclei for all GelMA-GNRs hybrids and pure GelMA constructs. 3C) 3D Z-stack fluorescent image (1 mg/mL GelMA-GNR) showing formation of cardiac tissue layer on top of hybrid construct.

FIG. 4A illustrates representative Immunostained images showing expression level of sarcomeric α-actinin (green), troponin I (green), and connexin 43 (red) for GelMA and 1.5 mg/mL GelMA-GNR hydrogel constructs. Images shows more organized and homogeneously distributed expression of cardiac-specific markers in hybrid constructs in comparison to pristine one.

FIG. 4B illustrates Z-stack (top-view) fluorescent images of expression of integrin β-1 (red) adhesion marker, across all hybrid and pristine hydrogel constructs. Scale bars represent 50 μm. DAPI (blue) represents stained nuclei. Considerable increase in expression level of integrin β-1 demonstrating highly packed and interconnected organization of cardiomyocytes in 1 and 1.5 mg/mL GelMA-GNR constructs.

FIGS. 5A-5C: 5A) Synchronous beating pattern (beats per minute-BPM) of cardiomyocytes from day 3 to day 7 of culture depicting robust and increasing beating behavior in GelMA-GNR hybrids with high GNRs concentrations (1 and 1.5 mg/mL). Statistically significant higher number of construct contraction (BPM) were observed between highly concentrated hybrids and 0.5 mg/mL GelMA-GNR and pure GelMA hydrogels (*p<0.05); 5B) beating signal pattern graphs represent more uniform and synchronous contraction behavior within 1 and 1.5 mg/mL GelMA-GNR, in contrast with 0.5 mg/mL hybrid and pristine GelMA hydrogels. 5C) Optical images of a detached centimeter scale hybrid hydrogel tissue (1.5 mg/mL GelMA-GNR) displaying contraction (white arrows) of the whole hydrogel construct by contractile cardiomyocytes.

FIG. 6 illustrates a schematic showing the GNRs synthesis steps by using a seed-mediated method.

FIG. 7A illustrates TEM micrographs of very thin layer (white circles) of 0.5 mg/mL GelMA-GNR hybrid hydrogels demonstrating GNRs (white arrows) are embedded within hydrogel.

FIG. 7B illustrates TEM micrographs of very thin layer (white circles) of 1 mg/mL GelMA-GNR hybrid hydrogels demonstrating GNRs (white arrows) are embedded within hydrogel.

FIG. 7C illustrates TEM micrographs of very thin layer (white circles) of 1.5 mg/mL GelMA-GNR hybrid hydrogels demonstrating GNRs (white arrows) are embedded within hydrogel.

FIGS. 8A-8C illustrate AFM based on nanoindentation on GelMA-GNR and pure GelMA. Force-indentation curves were carried out in 4×4 grids (force-volume mode). 8A) Maps of Young's moduli for different indentation depths. The force indentation curves were fitted in 1 μm segments (0-1 μm, 1-2 μm, and 3-4 μm indentation depths) and for each segment and curve the Young's modulus was determined. 8B) Cross section of depth dependent fit for different gold concentrations. 8C) Average Young's moduli for the different samples (*p<0.05). (Scale bars: Vertical 1 μm; horizontal 20 μm).

FIG. 9 illustrates mean pore diameter size of hydrogel constructs (GelMA and GelMA-GNR) determining considerable decrease in pore's diameter in highly concentrated (1 and 1.5 mg/mL) hybrid hydrogel constructs, as compared to pure GelMA hydrogels (*p<0.05).

FIG. 10 illustrates phase-contrast optical microscope images of cultured cardiomyocytes on top of hybrid GelMA-GNR and pure GelMA hydrogels at day 1, 4, and 7 illustrating cell retention (day 1) and cell spreading over the culture period. Scale bars represent 250 μm.

FIG. 11 illustrates immunostaining z-tack (top-view) images for sarcomeric α-actinin (green), connexin 43 (red) and troponin I (green) for 1 mg/mL GelMA-GNR hybrids showing high expression and homogeneous distribution of cardiac-specific markers.

FIG. 12 illustrates a table of mean length and width of synthesized GNRs with average aspect ratios of 3.15.

### DETAILED DESCRIPTION

GelMA-Gold nanorod (GNR) hybrid hydrogels for cardiac tissue engineering applications. Gelatin methacrylate (GelMA) is a photocrosslinkable hydrogel comprised of dehydrated gelatin functionalized with methacrylate groups. GelMA is a biodegradable hydrogel with numerous cell binding sites within its structure, which makes it an excellent candidate for tissue engineering applications. We hypothesized that due to enhanced electrical properties of GelMA-GNR composite hydrogels, these constructs will have lower impedance ultimately leading to enhanced cell-cell electrical coupling and improved signal propagation. Further, the surface-exposed GNRs will increase the local roughness within the hybrid hydrogels, which consequently, improves cell adhesion and retention of the seeded cardiomyocytes. This electrically- and structurally-mediated cellular communications will promote cellular phenotype and result in formation of functional cardiac tissues. To evaluate these hypotheses, we performed critical material and biological studies to associate the impact of GNRs on the function of nanoengineered cardiac tissue patches.

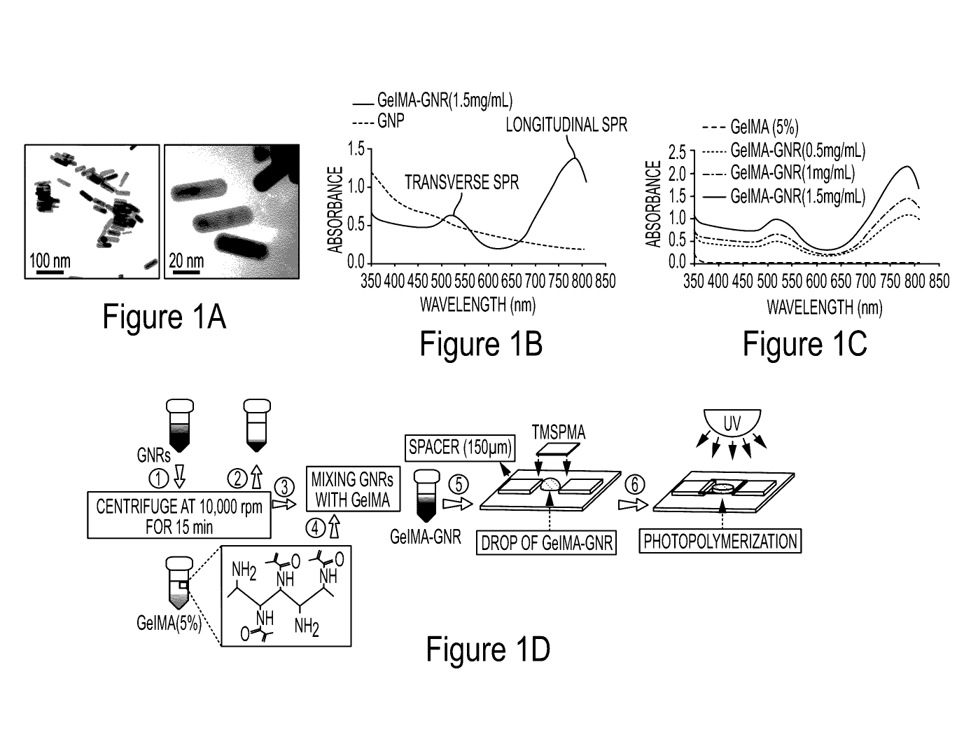

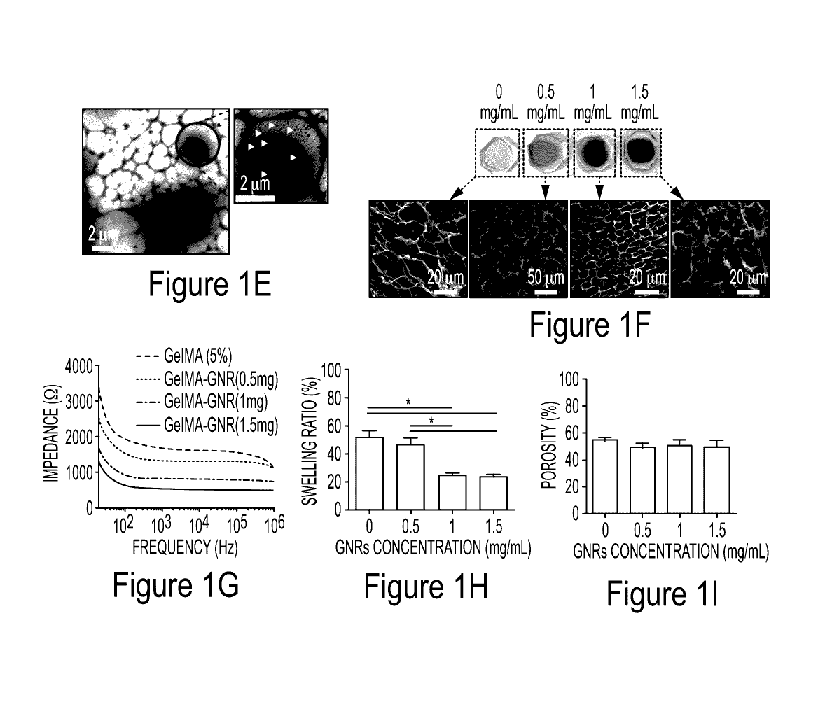

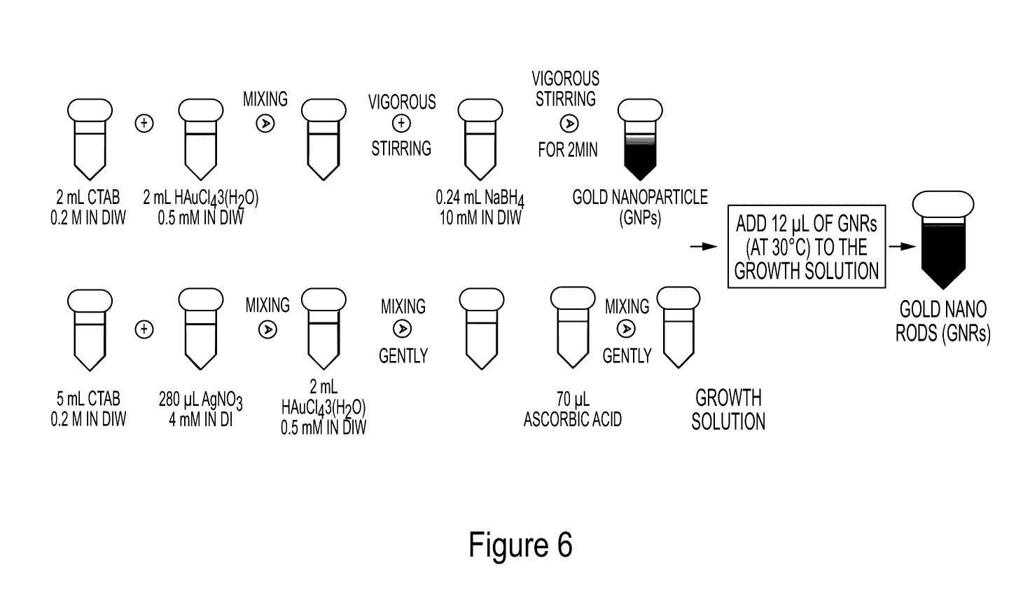

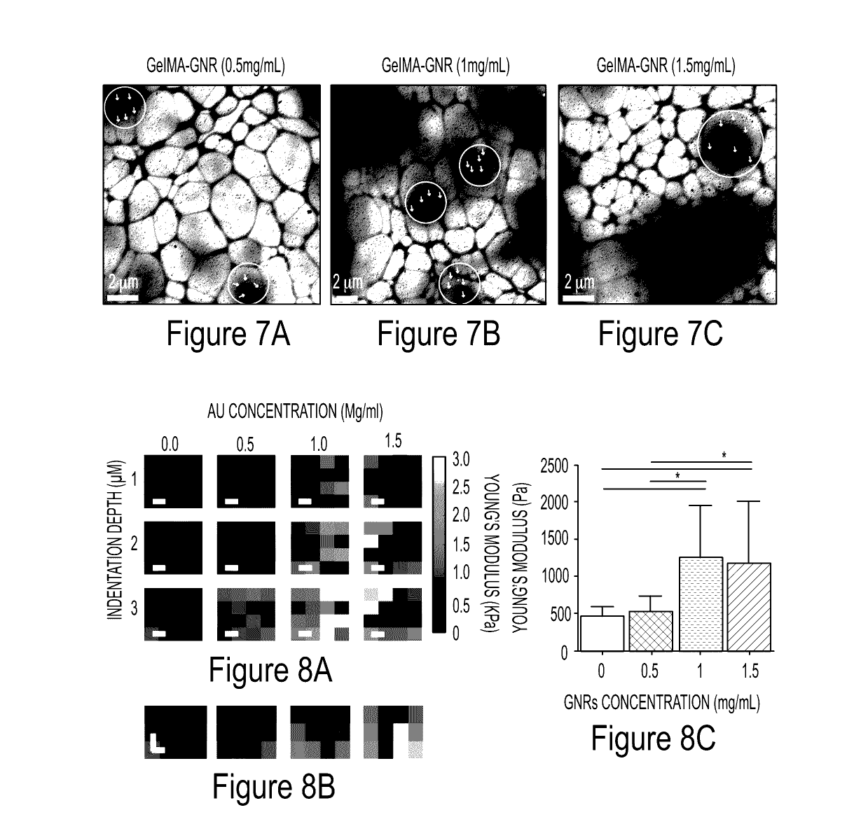

The following relates to fabrication and characterization of GelMA-GNR hybrid constructs. The gold nanorods were synthesized via using a seed-mediated growth method (See FIG. 6). TEM micrographs (FIG. 1A) demonstrated the rod-like shape of the gold nanomaterials; FIG. 1A shows TEM micrographs of synthesized GNRs. FIG. 1B shows the UV-Vis absorption spectrum illustrating the longitudinal (810 nm) and transverse (530 nm) surface plasmon resonance (SPR) 58 wavelength peaks of the GNRs. These results confirmed successful fabrication of GNRs consisted with previously published studies. Furthermore, the aspect ratios of the synthesized GNRs were calculated to be ˜3.15 as shown FIG. 12. The GelMA-GNR prepolymer solutions were sonicated to prepare a well-mixed mixture. All GelMA-GNR prepolymer solutions (0.5, 1, and 1.5 mg/mL) exhibited similar transverse and longitudinal wavelength peaks (FIG. 1C), confirming that the GNRs did not break into smaller sizes during the sonication process. The GelMA-GNR hybrid hydrogels (150 μm thick) were formed through UV photopolymerization, as shown schematically in FIG. 1D. The TEM images (FIG. 1E; FIG. 7A-7C) of a very thin layer of the GelMA-GNR constructs confirmed that GNRs were embedded within the GelMA hydrogel (white arrows). Moreover, the SEM images (FIG. 1F) of lyophilized pure and hybrid hydrogels revealed a similar macrostructure as well as homogeneous distribution (without creation of any agglomerations) of GNRs within all the hydrogel constructs. Thus far, the represented results indicated successful fabrication of pure GelMA and GelMA-GNR hybrid constructs with different GNRs concentration.

To evaluate the electrical conductivity of hybrid hydrogel constructs, impedance analysis was performed. FIG. 1G represents GelMA-GNR hybrids with high concentrations of nanorods (1 and 1.5 mg/mL) exhibited relatively low electrical impedance as compared to pure GelMA at physiologically-related frequencies. This low electrical impedance can be associated to resistive current through the bridging GNRs, suggesting that high concentration of GNR improves the electrical conductivity of the hybrid hydrogel. Such properties have been shown to facilitate the action potential propagation between cardiomyocytes, which consequently leads to enhanced electrophysiologically cell-cell coupling.

The Young's modulus (stiffness) as a representative of mechanical properties of hydrogels was measured with atomic force microscopy (AFM) based nanoindentation to investigate the constructs' capability for enduring compressive force, generated by the cardiac cells. The samples were indented in 4×4 grids up to a maximum depth of 4 μm and analyzed in 1 μm sections. FIG. 8 a) and b) show the distribution of local Young's moduli. The heterogeneity and stiffness augment with increasing GNR concentration. FIG. 8c) shows the average Young's moduli for indentation depths of 3-4 μm confirming a significant increase in elastic modulus of GelMA-GNR hybrids up to ˜1.3 kPa for 1 mg/mL GNRs in comparison to ˜450 Pa for pure GelMA. The quantified data herein for pure GelMA construct (150 thickness, 6 seconds UV exposure) relatively correlates to similar studies for a 1 mm thick hydrogel construct with 60 second UV exposure. The improved mechanical stiffness of hybrid hydrogels can be attributed to increased structural integrity caused by electrostatic interaction between positively charged gold nanorods with negatively charged amine groups of GelMA backbone. Also, gold nanorods acted as reinforcing fillers and thus induced mechanical enhancement. Despite the fact that incorporation of GNRs improved mechanical stiffness of pure GelMA hydrogel constructs up to 1.3 kPa, this value is still considerably lower than the human native myocardium stiffness during contraction cycle. However, similarly reported cardiac patches with low Young's modulus have demonstrated the suitability of these scaffolds, in terms of mechanical robustness, to accommodate cultured cardiomyocytes contractile force.

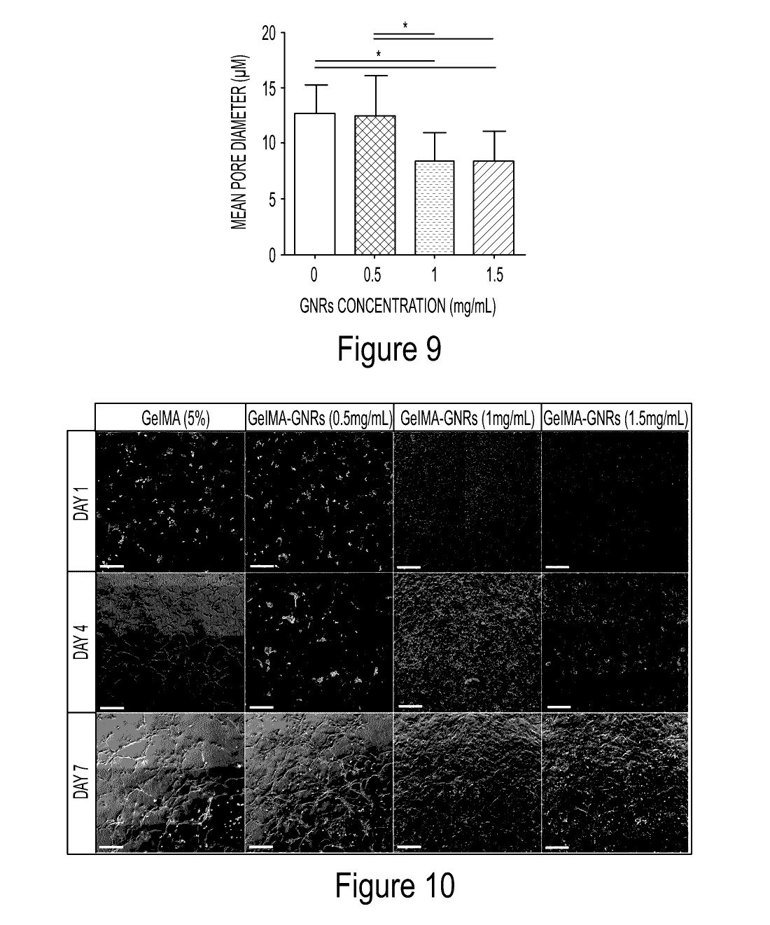

Swelling degree and porosity are the crucial characteristics of hydrogel-based scaffolds, directly influencing nutrient and waste exchange, specifically in the case of cell encapsulation, as well as cell ingrowth within the constructs. As illustrated in FIG. 1H, the incorporation of GNRs with high concentrations (1 and 1.5 mg/mL) within the GelMA hydrogel led to a statistically-significant decrease in the swelling ratio, from 51.72±4.85% for pure GelMA to the minimum of 23.58±2.24% for the hybrid GelMA-GNRs constructs (1.5 mg/mL). This decrease can be associated to the smaller pore size (See FIG. 9) of hybrid hydrogels (8.59±2.35 and 8.46±2.7 μm for 1 and 1.5 mg/mL GNRs concentration, respectively) compared to the pure GelMA (12.78±2.52 μm), which ultimately lead to a decrease in water content. Although incorporation of GNRs caused a decrease in pore size, nanoscale void structure still is available for nutrient and gas exchange within the all the hydrogel constructs. In addition, FIG. 1I displays that nanoscale GNRs did not notably affect the porosity percentage of the hydrogel construct, which ensures cellular infiltration.

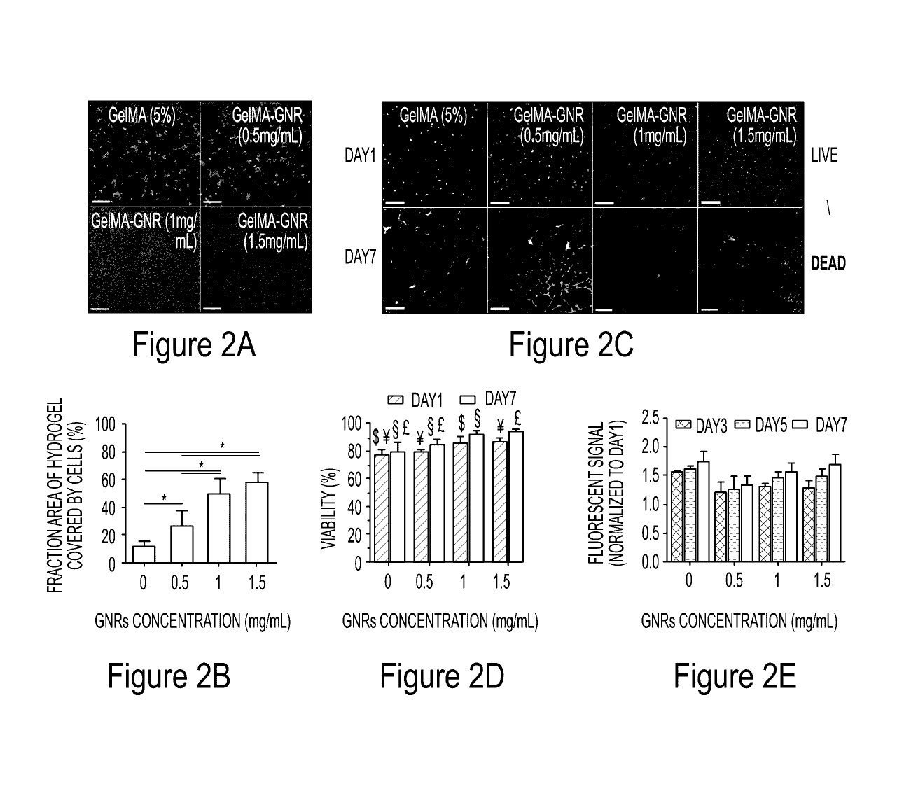

Assessments of cardiomyocytes retention, survival, and metabolic activity. To evaluate the capability of the nanoengineered hydrogel constructs to provide a proper substrate for cells adhesion and spreading, the fraction area of constructs which was covered by seeded cardiomyocytes (day 1) was quantified as an indicator for cell retention. The phase-contrast images (FIG. 2A) of the GelMA-GNR hybrid hydrogels demonstrated higher number, more packed and homogeneous distribution of adhered cardiomyocytes, contrary to discrete and agglomerated cell adhesion pattern in the pure GelMA hydrogel. Furthermore, FIG. 2B indicates a significant increase in cell retention as a function of GNRs concentration, from 11.71±3.67% for pure GelMA to 26.81±11.17%, 49.92±11.19%, and 58.71±6.54% for 0.5, 1, and 1.5 mg/mL GelMA-GNR hybrids respectively. This major dissimilarity between cell adhesion affinity of GelMA and GelMA-GNR hydrogels can be related to the difference in number of cell anchoring points on the surface of the constructs. In other words, exposed GNRs on the surface of hybrids acted as the excess cell adhesion sites, as well as the provided cell attachment motifs by gelatin composition structure, improving cell attachment and spreading, and consequently, reducing the rate of cell death. Such properties makes GelMA-GNR superior constructs as compared to previously reported gold-impregnated scaffolds for cardiac tissue engineering. For instance, Dvir et al. (Dvir, T.; Timko, B. P.; Brigham, M. D.; Naik, S. R.; Karajanagi, S. S.; Levy, O.; Jin, H.; Parker, K. K.; Langer, R.; Kohane, D. S. Nanowired three-dimensional cardiac patches. Nature Nanotechnology 2011, 6, 720-725) incorporated gold nanowires within an alginate hydrogel, which typically is not an adhesive substrate for cell spreading. In addition, You et al. (You, J.-O.; Rafat, M.; Ye, G. J. C.; Auguste, D. T. Nanoengineering the Heart: Conductive Scaffolds Enhance Connexin 43 Expression. Nano Letters 2011, 11, 3643-3648) embedded gold nanoparticles within a sophisticated thiol-HEMA/HEMA scaffold, but due to the cell-repellant nature of HEMA, the constructs needed to be treated with fibronectin prior to cell seeding to increase cell adhesion.

Cell viability (FIG. 2C, D; FIG. 10) was tested at days 1 and 7 of the culture in order to provide a comprehensive (seeding to the end culture time-point) investigation on cardiomyocytes survival on the fabricated hybrid hydrogel constructs. FIG. 2D illustrates cardiomyocytes viability increased significantly from 78±3.6% for pure GelMA hydrogel to 87.66±3.21% for 1 mg/mL and 88.66±1.52% for 1.5 mg/mL GelMA-GNR hybrid hydrogels at day 1. The viability values for GelMA and hybrids at day 7 exhibited cultured cardiomyocytes maintained a considerable high level of survival over the whole duration of culture. Next, we determined cellular proliferation as measured by Alamar Blue fluorescent signals at days 3 (FIG. 2E), 5 (FIG. 2F), and 7 (FIG. 2G) of culture normalized to day 1. Across all pure and GelMA-GNR hydrogels, no statistically significant cellular proliferation was observed. However, cultured cells exhibited an increase in cellular proliferation for each construct on day 5 and 7 as compared to day 3 of culture. Despite the fact that cardiomyocytes do not demonstrate proliferative capability, this rise can be attributed to the proliferation of few residence cardiofibroblast, which are usually present during cell isolation process. Nevertheless, Martinelli et al. (Martinelli, V.; Cellot, G.; Toma, F. M.; Long, C. S.; Caldwell, J. H.; Zentilin, L.; Giacca, M.; Turco, A.; Prato, M.; Ballerini, L., et al. Carbon Nanotubes Promote Growth and Spontaneous Electrical Activity in Cultured Cardiac Myocytes. Nano Letters 2012, 12, 1831-1838) reported that conductive nanomaterials, carbon nanotubes, stimulate cardiomyocytes proliferation.

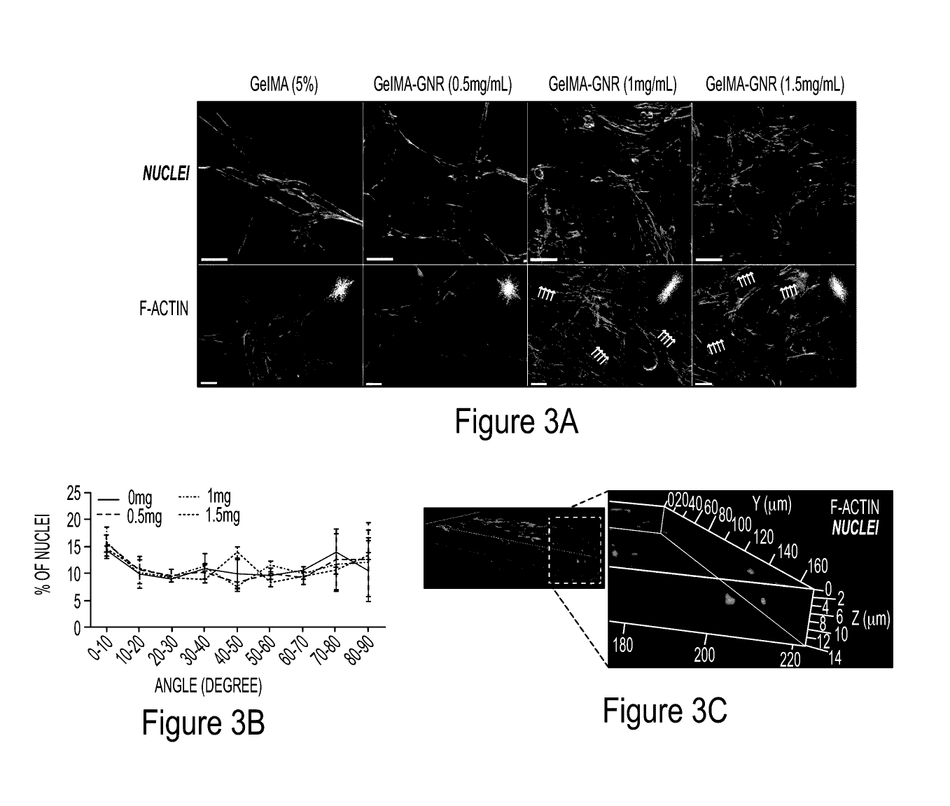

Cytoskeleton organization and formation of cardiac tissue layer. In order to investigate cytoskeletal organization and the morphology of formed tissue, cardiomyocytes were stained for F-actin fibers at day 7 of culture. F-actin fibers were highly polymerized (FIG. 3A) on GelMA-GNR hybrids (1 and 1.5 mg/mL), in contrast with the lower expression of microfilaments on the pure GelMA hydrogels. As is shown in FIG. 3A, within the hybrid GelMA-GNR hydrogels with 1 and 1.5 mg/mL GNRs concentrations, F-actin fibers were organized in a highly uniform and intact profile with a local alignment, while they were localized in an arbitrary arrangement in the pure GelMA hydrogel. Furthermore, FFT images (FIG. 3A insets) confirmed local alignment of F-actin fibers in the hybrid constructs with higher GNRs concentrations, in comparison with randomly distributed fibers in the pure GelMA hydrogel. Also, FIG. 3B illustrates nuclei alignment distribution from 0 to 90 degrees in a 10 degrees increment manner. Due to unpatterned geometry of pure and hybrid hydrogel constructs, approximately the same percentage of nuclei alignment (11.03±1.87% for pure GelMA, 11.03±2.25% for 0.5 mg/mL, 11.07±2.31% for 1 mg/mL, and 11.07±2.36% for 1.5 mg/mL GelMA-GNR) was within the hydrogel constructs.

The elongated and packed cellular organization within the hybrid GelMA-GNR hydrogels led to the formation of a uniform and interconnected tissue layer. The top-view of a z-stack fluorescent image (FIG. 3B) and phase-contrast images (FIG. 10) demonstrated the formation of a tissue layer on top of hybrid constructs over 7 days of culture. Thus, improved cell adhesion and spreading, through more cell anchoring sites, in the gold-embedded hydrogels, particularly those with higher concentration of GNRs, consequently led to formation of an intact cardiac tissue layer.

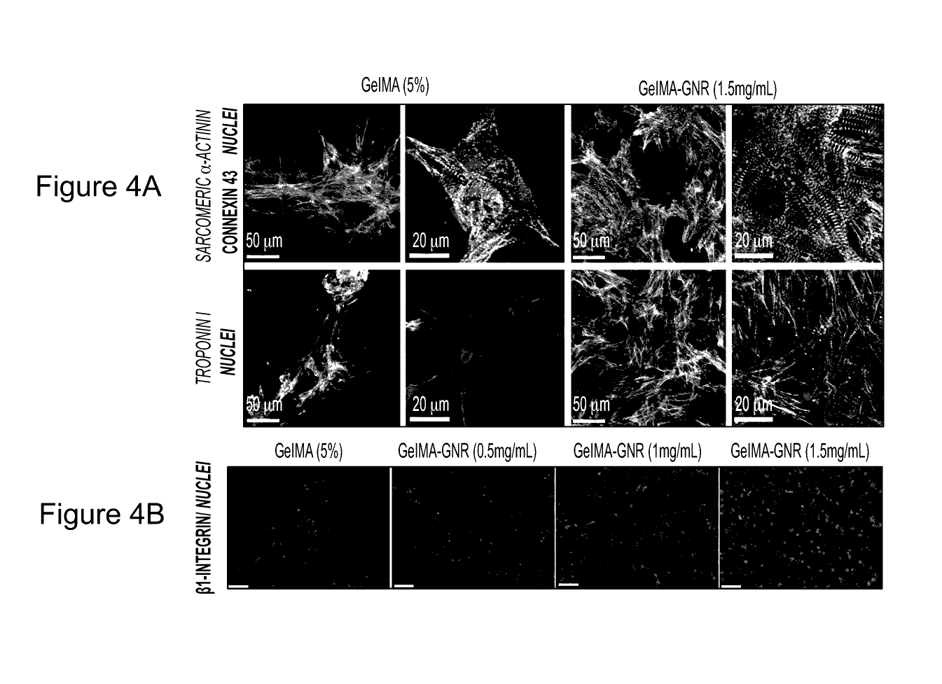

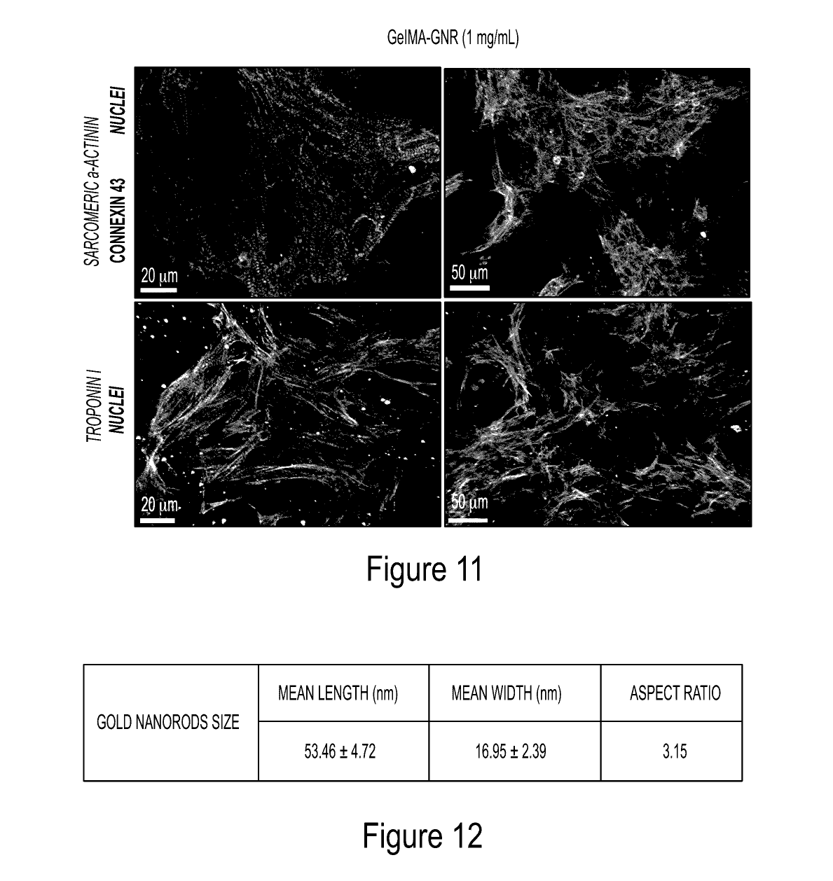

Evaluation of cardiac-specific and cell adhesion markers. Cardiac-specific markers (sarcomeric α-actinin, connexin 43, and troponin I) were immunostained at day 7 of culture to assess the phenotype of the cultured cardiomyocytes. Sarcomeric α-actinin and troponin I are two particular proteins which are a part of the actin-myosin contraction complex. Immunostaining images (FIG. 4A; FIG. 1I) represented a notably higher expression of sarcomeric α-actinin (green) with more uniform and extended structures in 1 and 1.5 mg/mL GelMA-GNR hybrids in comparison to pure GelMA. Additionally, a one-directionally alignment pattern was observed for most of expressed sarcomeric structures in the case of hybrid constructs, contrary to the undefined and discrete expression patterns in the pure GelMA hydrogels. Furthermore, cardiomyocytes expressed (FIG. 4A; FIG. 11) a considerable level of troponin I (green) in the hybrids with 1 and 1.5 mg/mL concentration of nanorods. In other words, formed tissue on the GelMA-GNRs, with high amount of impregnated GNRs, exhibited more uniform and organized myofilament assembly. Connexin 43 (Cx43), a gap junctions responsible for cell-cell electrical signal coupling, was also highly expressed in GelMA-GNR hybrid scaffolds as compared to pure GelMA (FIG. 4A) 51, 71-73. Cx43 gap junctions were homogeneously dispersed within the cardiomyocytes cultured on 1 and 1.5 mg/mL GelMA-GNR hybrids, which led to enhanced cell-cell communication.

Providing a surface with high affinity for cell adhesion and spreading is an important characteristic for an ideal hydrogel cardiac patch. A hydrogel with improved surface features can induce cell-cell mechanical and electrical coupling, which eventually results in the formation of a highly functional tissue construct. Moreover, integrin-based adhesive junctions are among the crucial components indicating cell-matrix interactions. Therefore, we stained integrin-β1, a transmembrane protein mediator, to analyze cellular adhesion on the nanoengineered hydrogels. FIG. 4B shows immunostaining images of integrin-β1 at day 7. Distinguished upregulation of integrin-β1 was observed as a function of GNR concentration.

Overall, cultured cardiomyocytes on GelMA-GNR hybrids (specifically 1 and 1.5 mg/mL nanorods) demonstrated more mature phenotypes and organized structures compared to pure GelMA hydrogel. In fact, nanoengineered hybrid constructs with enhanced electrical conductivity facilitate cell-cell electrochemical coupling, which eventually leads to the formation of a functional tissue. To further investigate the influence of conductive GelMA-GNR hybrid hydrogels on tissue functionality, we analyzed the beating behavior of the cultured cardiomyocytes.

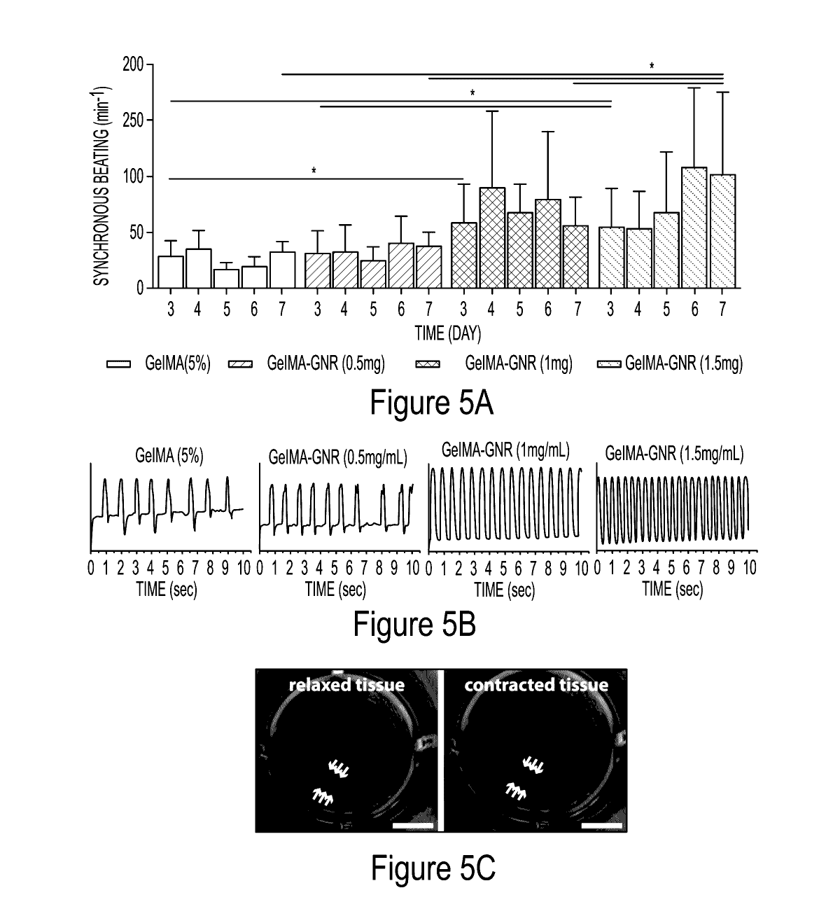

Beating behavior analysis of cultured cardiomyocytes. Beating behavior (as a number of synchronous beats per minute (BPM)) of hydrogel constructs was analyzed through capturing real-time video microcopy of beating tissues from day 3 to day 7 of culture. Cardiomyocytes started beating in a spontaneous, synchronous manner, as they reached together and created an interconnected cell network as a function of GNR concentration. FIG. 5A displays average number of BPM for all of the hybrid and pure hydrogel constructs. As can be observed at day 3, hybrid tissue constructs with 1 and 1.5 mg/mL GNRs concentrations demonstrated a significantly higher number of synchronous beatings as compared to pure GelMA. By day 7, GelMA-GNR (1.5 mg/mL) hydrogels showed a noteworthy increase in number of beats (102.3±72.65 BPM), while the 1 and 0.5 mg/mL GelMA-GNR hybrids and pure GelMA revealed 56.92±24.6, 38.32±11.38, and 33.33±9.62 BPM respectively.

In addition, beating frequency analysis (FIG. 5B) illustrated more uniform, stable, and robust beating behavior for GelMA-GNR hybrids (1 and 1.5 mg/mL) in comparison to pure GelMA hydrogel. This robust beating behavior can be attributed to the enhanced electrophysiological characteristics of formed tissue on nanoengineered hydrogels. Additionally, to examine the capability of formed cardiac tissue to contract the whole hydrogel construct, we detached 2 samples of hybrid hydrogels (1 and 1.5 mg/mL GelMA-GNR) at day 5 from the TMSPMA glass slide and suspended the tissue constructs in culture media (FIG. 5C). A suspended beating tissue sheet (centimeter scale) was created using the 3D hydrogel construct. Apparently, it is evident that created cardiac tissue on top of GelMA-GNR hydrogel constructs was highly functional in a way to bend the 150 μm thick construct.

Although similar observations for highly stiff CNT-embedded GelMA hydrogels were reported, however cytotoxicity and high UV absorption of CNTs remain as considerable issues for future cardiac tissue engineering applications. Specifically, high UV absorbance of CNTs interferes with photoinitiatior excitation, and consequently influences hydrogel crosslinking. This phenomenon requires high levels of UV irradiation for proper hydrogel crosslinking, which causes limitations for fabrication of a 3D thick tissue construct. On the other hand, GNRs with low UV absorption (FIG. 1B, C) and minimized cytotoxicity enable us to fabricate thick cardiac patches (e.g. 150 μm).

In this study, we investigated GNR-embedded in GelMA hydrogel as a potential cardiac patch for myocardial regeneration and repair. GNRs with aspect ratio 3.15 (16.95±2.39 nm width and 53.46±4.72 nm length) were synthesized and added to GelMA prepolymer solution, followed by UV crosslinking to fabricate hybrid GelMA-GNRs constructs (150 μm thick). The GelMA-GNR hybrids exhibited enhanced surface properties, which led to higher cellular retention. GNRs acted as cell adhesion sites and improved cell attachment. This mediated cell-cell coupling and upregulated the expression of F-actin fibers, adhesive junction proteins (integrin β1), and cardiac specific markers including sarcomeric α-actinin and troponin I, resulted in the creation of a healthy functional tissue. The formed tissue represented an organized, packed, and uniform architecture while maintaining a high level of cellular viability over 7 days of culture. The impregnated GNRs created a conductive network and bridged the insulated gaps within the hydrogel structure. The conductive construct can facilitate cell-cell electrochemical signaling and action potential propagation, which consequently leads to improve tissue function. Also, the expression of Cx43 gap junctions was notably upregulated as a function of nanorods concentration, which prompted intracellular electrochemical communication. These enhancements eventually gave rise to higher performance of cardiac tissue constructs and improved tissue functionalities, such as beating. GelMA-GNR hybrids (1 and 1.5 mg/mL) illustrated more robust and synchronous beating rate with higher stability in comparison to the pure GelMA hydrogel. In conclusion, the nanoengineered GelMA-GNR hybrid hydrogels exhibited superior characteristics compared to the pure GelMA hydrogel, and induced the formation of a highly functional cardiac patches.

Experimental Methods

Materials. Gold (III) chloride trihydrate (HAuCl4) (>99.9%), sodium borohydride (NaBH4) (>99%), hexadecyltrimethylammoniumbromide (CTAB) (>99%), silver nitrate (AgNO3) (>99%), and L-Ascorbic acid (>98%) were purchased from Sigma-Aldrich and used without further purification. Gelatin (Type A, 300 bloom from porcine skin), methacrylic anhydride (MA), 3-(trimethoxysilyl) propyl methacrylate (TMSPMA), and 2-hydroxy-1-(4-(hydroxyethoxy) phenyl)-2-methyl-1-propanone (the photoinitiatior) all were obtained from Sigma-Aldrich. Deionized water (DIW) (18MΩ) was used for the all GNR fabrication processes.

Gold nanorod (GNR) synthesis. GNRs were synthesized (FIG. 6) by using a previously established seed mediated growth method. First, HAuCl4 (0.5 mM) was dissolved in DIW (Distilled water) and mixed with a 2 mL aqueous solution of CTAB (0.2 M), which turned the color of the solution to deep yellow. Then, 240 μL of ice-cold NaBH4 was added all at once to the solution under stirring and was kept stirring for 2 min. The color of the solution changed from deep yellow to brownish yellow immediately, which indicates gold nanoparticle (seed solution) formation. A seed solution contains gold nanoparticles (GNPs) with a mean diameter of less than 4 nm.

The growth solution was prepared by adding 1.12 mL AgNO3 (4 mM in DIW) to 20 mL CTAB (0.2 M in DIW) followed by the addition of a 20 mL aqueous solution of HAuCl4 (1 mM), which created a deep yellow solution. To this solution, 280 μL ascorbic acid (13.88 mg in 1 mL DIW) was added very gently, and immediately the solution turned colorless. The temperature was kept at 25° C. during the processes.

Finally, a 48 μL aliquot of seed solution was poured into the growth solution at 30° C. and the color of solution changed to brownish red over a period of half an hour, which indicated the formation of GNRs. To attain longer GNRs, the solution was kept overnight at 30° C. The solution contained 99% GNRs with an aspect ratio (length/width) of ˜3.15 (FIG. 12). Before any further experimentation, the GNR colloid was centrifuged 12,000 rpm for 10 min and washed 2 times with DIW to remove the excess CTAB.

Gelatin methacrylate synthesis. GelMA was synthesized as previously described protocol 54. In brief, type A gelatin (Sigma-Aldrich, USA) (10% w/v) was fully dissolved in Dulbecco's phosphate buffered saline (DPBS) at 50° C. Then, MA (8% v/v) was added drop-wise to the gelatin solution and stirred for 3 h at 50° C. To stop the methacrylation reaction, the solution was diluted 5 times by adding DPBS (50° C.). The final solution was then poured into dialysis tubes (12-14 kDa molecular weight cutoff) and kept at 45° C. in DIW under stirring for 7 days to eliminate the unreacted MA and salt. Dialyzed solution was passed through a 0.2 μm filter and then lyophilized for 7 days to obtain the white GelMA foam.

Preparation of GelMA-GNR hybrid hydrogels. Primarily, the photoinitiatior (0.5% w/v) was completely dissolved in DPBS, and then to this solution, lyophilized GelMA foam (5% w/v) with high degree of methacrylation (96.41±1.54%) was added and kept at 80° C. until a clear solution was achieved. Second, certain amounts of centrifuged GNRs (0.5, 1 & 1.5 mg/mL) were mixed with GelMA prepolymer followed by sonication for 1 h to obtain a homogeneous mixture. To prepare hybrid constructs (FIG. 1A), 15 μL of the GelMA-GNR mixture was placed between two 150 μm tall spacers and covered by glass slides coated with TMSPMA. The constructs were formed via photopolymerization by UV light (800 mW, 360-480 nm) for 6, 8, 25, and 35 seconds exposure time for 0, 0.5, 1, and 1.5 mg/mL GNR concentrations respectively.

Characterization of GNR and GelMA-GNR hybrid hydrogels. GNR micrographs were obtained by using transmission electron microscopy (TEM) (Philips CM200-FEG, USA) operating at an accelerating voltage of 200 kV. Macroporous structures of hydrogel constructs were evaluated by means of scanning electron microscopy (SEM) (XL30 ESEM-FEG, USA). To prepare samples, swollen hydrogels were placed in liquid nitrogen followed by freeze-drying, and then samples were coated with Au/Pd (4 nm). Five SEM images were selected to analyze the porosity and average pore size measurements using NIH ImageJ software. To measure the mechanical stiffness (Young's modulus) of hydrogel constructs, 150 μm thick swollen hydrogels in DPBS were tested by an atomic force microscopy (AFM) (MFP-3D AFM, Asylum Research) with silicon nitride tips (MSNL, Bruker). Three samples were used for each GNR concentration and the contact model for a cone indenter was used REF. For impedance analysis, hydrogel constructs were located between two indium tin oxide (ITO) coated glass slides (Sigma-Aldrich) with an AC bias sweeping (impedance device name) from 20 Hz to 1 MHz. Three samples were analyzed per each GNRs concentration. To evaluate swelling behavior of pristine and hybrid hydrogel constructs, 10 mm radius disc-shape (150 μm height) hydrogels were prepared and immediately soaked in the DPBS and locate in 37° C. for 24 hours. Constructs were blotted with KimWipe very gently to remove the residual DPBS and the weight values were recorded. Afterwards, the swollen hydrogels were immersed in liquid nitrogen, followed by lyophilization. The swelling ratio defined as below (eq. 1):

$$Swelling ratio = \frac{{M}_{wet} - {M}_{dry}}{{M}_{wet}} \times 100 \qquad ( eq . 1 )$$

where, M_{wet} is mass of swollen and M_{dry} is mass of lyophilized hydrogel. For each GNRs concentration, three identical samples were selected.

Ventricular cardiomyocytes isolation and culture. Cardiomyocytes were harvested from the ventricular region of 2 day old neonatal rats based on previously developed protocol accepted by the Institution of Animal Care at Arizona State University 7. After isolation, the cardiomyocytes were separated from cardiofibroblasts by pre-plating the cells suspension for 1 h. Before seeding cardiomyocytes, hydrogel constructs were soaked 2 times with 10 min intervals in 1% (v/v) penicillin-streptomycin (Gibco, USA) in DPBS and then washed 2 times in 10 min periods in the cardiac culture medium containing Dulbecco's modified eagle medium (DMEM) (Gibco, USA), 10% fetal bovine serum (FBS) (Gibco, USA), 1% L-Glutamine (Gibco, USA), and 100 units/mL penicillin-streptomycin. Cardiomyocytes were seeded on top of disk-shape constructs (diameter in height, 10 mm×150 μm; 7.5×105 cells/well) and were cultured in the cardiac specific culture media for 7 days under static condition (no electrical stimulation).

Characterization of survival, retention metabolic activity and phenotype of the cardiomyocytes. Cardiomyocytes viability was determined using a Live/Dead assay (Life technologies, USA) based on manufacturer's instruction. Triplicates samples were used for each hydrogel construct and 3 individual areas were selected within each replicate. Fluorescent images were acquired by using a fluorescent microscope (Zeiss Observer Z1) and the quantification was processed by ImageJ software. The viability was quantified as number of live cells divided by total number of cells. Cell retention was measured according to area fraction, using ImageJ software, one day upon seeding. Five phase contrast images were taken by utilizing an inverted light microscope for each sample (three samples for each hydrogel group). To examine metabolic activity of cells on the constructs, Alamar Blue assay kit (Invitrogen, USA) was used according to manufacturer's protocol at days 3, 5, and 7 of culture. Three samples were specified for each hydrogel construct and results were normalized with respect to day 1.

The immunocytochemistry technique was used to visualize expressed proteins. In the case of cardia-specific markers, including sarcomeric α-actinin, connexin, and troponin I, cardiomyocytes were fixed in 4% paraformaldehyde (PF) for 35 min followed by treatment with 0.1% Triton x-100 for 45 min at room temperature to permeabilize the plasma membrane. Then, cells were blocked in 10% goat serum for 2 h at room temperature. Afterwards, cardiomyocytes were stained with primary antibodies (1:100 dilution in 10% goat serum) and placed in a cold room (4° C.) for 24 h. After primary staining, samples were washed with DPBS and stained with secondary antibodies (Abcam, USA) comprising Alexa Fluor-594 (pseudo-colored with green) for sarcomeric α-actinin, Alexa Fluor-488 for troponin I and connexin (pseudo-colored with red) at a 1:200 dilution in 10% goat serum for 6 h. Eventually, cells were treated with 40,6-diamidino-2-phenyl indole dihydrochloride (DAPI) (1:10000 dilution in DPBS) for ˜18 min to stain the nuclei. For adhesion specific marker (integrin-β1) all staining steps were the same as cardiac markers except that the cell's membrane were not permeabilized. Alexa Fluor-488 secondary antibody was used to stain integrin-β1.

To assess the cytoskeleton, hydrogel constructs were stained for F-actin. Cells were fixed in PF and soaked in Triton x-100 (the same as cardiac-related proteins staining procedure), and were then blocked in 1% (v/v) bovine serum albumin (BSA) for 1 h. Finally, cardiomyocytes were stained (1:40 dilution in 1% BSA) with Alexa Fluor-488 phalloidin (Life technologies, USA) for 40 min, and counterstained with DAPI (1:10000 dilution) for ˜18 min. Z-stack fluorescent images were taken by a fluorescent microscope equipped with ApoTome2 (Zeiss, Germany) and analyzed by ImageJ (FFT built-in plugin). Cell's nuclei alignment was quantified similar to previously established procedure. Briefly, an ellipse (built-in plugin, ImageJ) was fitted to the nuclei (DAPI) and deviation angle from the main axis of ellipse with respect to the x-axis was determined. All alignment angles were normalized by subtracting from average angle of each image and presented in 10° increment spans. The spontaneous beating of cardiomyocytes was measured and monitored from day 3 to day 7 of culture. For each data point, 3 videos (30 sec long) were captured per each sample (9 replicates for each group of hydrogel construct). Also, beating frequency was obtained by using a custom written MATLAB code 17.

Statistical analysis. The data collected in this study were analyzed by means of a one-way and two-way ANOVA analysis methods and were reported as mean±standard deviation (SD). To determine a statistically significance difference between groups, we used a Tukey's multiple comparison test and we considered a P-value<0.05 to be significant. All the statistical analysis were performed by GraphPad Prism (v.6, GraphPad San Diego).

Using the various techniques discussed above, UV-crosslinkable gold nanorod (GNR)-incorporated gelatin methacrylate (GelMA) hydrogels were formed with improved electrical and structural properties for cardiac tissue engineering. Homogeneously dispersed GNRs enhanced electrical conductivity of insulated GelMA structure, facilitating signal propagation and cell-cell coupling within the hydrogel constructs. This electrically- and structurally-mediated cellular communications directly influenced cell phenotype and eventually led to enhanced tissue functionality. Specifically, cardiomyocytes in GelMA-GNR hybrids exhibited greater cell retention as well as maintained a high level of viability over the whole duration of culture. Furthermore, enhanced expression of integrin β-1, a transmembrane protein involved in cell adhesion, confirmed improved cell-matrix interaction on GelMA-GNR hybrid hydrogels. This increased cell adhesion and spreading affinity resulted in upregulation in expression of F-actin fibers with local alignment and uniform organization indicating the formation of a highly integrated tissue layer on the GNRs-embedded hydrogels. Considerable increase in expression of cardiac specific markers (sarcomeric α-actinin, troponin I, and connexin 43 gap junctions) as a function of nanorods concentration was observed on the hybrid hydrogels. Particularly, intact sarcomeric α-actinin structures and homogeneously distributed connexin 43 gap junctions were formed in GelMA-GNR constructs. Notably, GelMA-GNR hybrids (1 and 1.5 mg/mL) demonstrated a robust synchronized tissue-level beating from day 3 to 7 of culture. The findings of this study indicated that highly functional cardiac patches with superior electrical and structural properties could be developed using nanoengineered-GelMA-GNR hybrid hydrogels.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A cell construct comprising:
GelMA-GNR hybrid hydrogels formed of GelMA and gold nanorods; and
where the gold nanorods are embedded in the GelMA hydrogel.

2. The cell construct as recited in claim 1, wherein the gold nanorods have a rod shape.

3. The cell construct as recited in claim 1, wherein the GelMA-GNR hybrid hydrogels having a surface with high affinity for cell adhesion and spreading.

4. A cardiac tissue patch assembly for myocardial regeneration and repair, the assembly comprising:
a cardiac patch including GelMA-GNR hybrid hydrogels formed of GelMA and gold nanorods, where the gold nanorods are embedded in the GemMA hydrogel.

5. The cardiac tissue patch assembly as recited in claim 4, wherein the hydrogels have a high concentration of GNRs of about 1-1.5 mg/mL.

6. The cardiac tissue patch assembly as recited in claim 4, wherein the patch has a thickness of about 150 μm.

7. The cardiac tissue patch assembly as recited in claim 4, wherein the patch has a stiffness up to 1.3 kPa.

8. A method of making a cardiac tissue patch assembly for myocardial regeneration and repair, the method comprising applying UV irradiation to a solution comprising gold nanorods and GelMA prepolymer to crosslink the GelMA-GNR.
